(19) United States
(12) Patent Application Publication
(10) Pub. No.: US 2021/0381032 A1
(43) Pub. Date: Dec. 9, 2021

(54) Rapid PCR Methodology

(71) Applicant: Longhorn Vaccines and Diagnostics, LLC, Bethesda, MD (US)

(72) Inventors: Luke T. Daum, San Antonio, TX (US); Gerald W. Fischer, Bethesda, MD (US)

(73) Assignee: Longhorn Vaccines and Diagnostics, LLC, Bethesda, MD (US)

(21) Appl. No.: 17/289,279

(22) PCT Filed: Oct. 31, 2019
(86) PCT No.: PCT/US2019/059150

§ 371 Date: Apr. 28, 2021

Related U.S. Application Data
(60) Provisional application No. 62/882,831, filed on Aug. 5, 2019.
(60) Provisional application No. 62/773,566, filed on Nov. 30, 2018.
(60) Provisional application No. 62/758,173, filed on Nov. 9, 2018.

(51) Int. Cl. C12Q 1/689; C12Q 1/6816; C12Q 1/6851; C12Q 1/70

(52) U.S. Cl. CPC C12Q 1/689; C12Q 1/6816; C12Q 2600/156; C12Q 1/701; C12Q 2600/106; C12Q 1/6851

(57) ABSTRACT

Disclosed is an enhanced method for rapid and cost-effective analysis of sequences of a microorganism by qPCR. These methods identify allelic variation, SNPs, and genetic mutations of a particular gene such as those responsible for conferring resistance or sensitivity to an antibiotic, chemotherapy, or another chemical compound. By selection of appropriate gene regions, mutation loci that confer resistance to key antibiotics can be identified by qPCR. Additionally, the approach can identify heteroresistant strains, e.g., populations of strains from a sample that contain both mutation and wild-type nucleotides. By selecting appropriate that bind efficiently to the area of mutation can identify resistance conferring mutations. Methods are useful to sequences derived from viral agents, such as influenza virus, bacterial agents, such as tuberculosis bacteria, and cancer cells.

### REFERENCE TO RELATED APPLICATIONS

This application claims priority to U.S. Provisional Application No. 62/882,831 filed Aug. 5, 2019, U.S. Provisional Application No. 62/773,566 filed Nov. 30, 2018, and U.S. Provisional Application No. 62/758,173 filed Nov. 9, 2018, the entirety of each of which is specifically incorporated by reference.

### BACKGROUND

### 1. Field of the Invention

This invention is directed to tools, compositions and methods for identifying genetic variations of a genome by rapid PCR methodologies and, in particular, to high throughput analysis of nucleic acids for rapidly identifying drug sensitivities of organisms.

### 2. Description of the Background

Mycobacterium tuberculosis (MTB), the causative agent for tuberculosis, is a highly transmissible bacterial pathogen with significant morbidity and mortality, particularly in HIV infected patients. Since 1997 tuberculosis has remained the leading cause of death in South Africa, a statistic linked to this country's growing HIV epidemic. Moreover, effective treatment measures in patients with active MTB have been exacerbated by increasing cases of multidrug resistance (MDR) and extensively drug-resistant (XDR) clinical isolates.

Microscopy remains the cornerstone for diagnosing MTB in many low resource areas of the world where both MTB and HIV are prevalent. However, many HIV infected patients with MTB are smear negative, and microscopy provides no information about antibiotic resistance. The emergence of multidrug-resistant (MDR) and extensively drug-resistant strains (XDR) has rendered standard MTB treatment regimens ineffective. According to one study, approximately 20% of TB patients in South Africa with HIV have MDR MTB. Rapid detection of MTB and initiating effective therapy is critical to decrease transmission and improve treatment outcome. The roll-out of Cepheid's Gene Xpert (Xpert) has improved MTB diagnosis and provides evidence of Rifampin resistance, but information about other drugs is not provided. Furthermore, it may not be feasible to place Xpert testing in many microscopy labs in low resource settings. The ability to efficiently ship sputum samples centrally for qPCR offers an opportunity to utilize highly trained staff and available infrastructure at central or regional laboratories.

MDR tuberculosis strains are resistant to first-line antibiotics rifampin (RIF) and isoniazid (INH), while XDR MTB strains are resistant to both RIF and INH as well as any fluoroquinolone and second-line injectable antibiotic drugs (e.g., amikacin, kanamycin or capreomycin). About 6% of all MTB cases are MDR strains and South Africa continues to report higher percentages of XDR cases each year. While 7% of patients infected with standard MTB strains succumb to infection, death rate rises to almost 50% with MDR tuberculosis. The emergence of antibiotic resistant MTB strains underscores an immediate need for rapid and highly accurate diagnosis, particularly in the developing countries of Africa. In addition, migratory populations make geographical surveillance and tracking of drug resistance strains more urgent.

Culture-based drug susceptibility testing (DST) for MDR strains is considered the gold-standard, but is time consuming (weeks to months), technically challenging and cost prohibitive, especially in resource limited countries. For example, the BACTEC MGIT 960 (Becton Dickinson Microbiology System, Silver Sparks NV, USA), is an automated continuously culture-based monitoring system that measures bacterial oxygen consumption and can perform DST using prepared kits which are available for susceptibility of strains to several antibiotics. DST results obtained with the BACTEC MGIT 960 yield reliable and reproducible but require handling of viable and potentially infectious cultures, ‘days to weeks’ or delay until results are available, specialized laboratory accommodations and high costs associated with the instrumentation and consumables.

In recent years, several nucleic acid-based assays for determining MTB drug resistance have been developed. One of the most popular commercially available diagnostic assays is the GenoType MTBDRplus Line Probe Assay (LPA) by Hain LifeScience. This test employs nucleic acid extraction, PCR amplification, probe hybridization and colorimetric visualization on lateral strips via an alkaline phosphatase reaction. LPA has been shown to be sensitive and specific, but there are several drawbacks. Sensitivity of the LPA for all resistance-associated mutations will most likely never reach 100% since many mutations that confer resistance have yet to be discovered. Another inherent limitation of the LPA is an inability to detect sample populations that contain a mixture of resistant and susceptible strains.

Real-time geographical surveillance of emerging MTB drug resistance would facilitate more appropriate treatment strategies (e.g., drug, antibiotic, chemical). Currently, available molecular methods such as the GenoType® MTBDRplus LPA and sequencing require specialized and expensive equipment and, moreover, results are not available for days. In addition, other microbes and cells mutate and rapid detection of key gene mutations is critical. Thus, there is a need for a rapid, standardized, cost-effective protocol for qPCR gene analysis of critical genes such as, for example, genes associated with MDR.

### SUMMARY OF THE INVENTION

The present invention overcomes disadvantages associated with current strategies and designs, and provides tools, compositions, methods to facilitate and simplify rapid qPCR techniques for drug resistance testing.

One embodiment of the invention is directed to rapid methods for detecting genetic variation within a target sequence of a genome of an organism. The method comprises: providing a pair of nucleic acid primers that span the target sequence and the target sequence comprises a conserved region of the genome; providing two nucleic acid probes, wherein each probe hybridizes to the target sequence, each probe is differentially labeled at each respective 5′-terminus and/or 3′-terminus and the sequence of a one probe differs from the sequence of an other probe by one nucleotide; combining the pair primers and the two nucleic acid probes with the target sequence forming a mixture; performing a polymerase chain reaction (PCR) of the mixture; detecting the labels; and determining the presence of genetic variation in the target sequence by the differential quantity of each label detected. Preferably the organism comprises a bacterium, such as Mycobacterium, a virus such as Influenza, a fungus, or a mammal. Preferably the target sequence comprises a segment of a gene or genome that confers drug resistance to the organism. For Mycobacteria, the gene comprises a rpoB gene and the sequences of the probes differ at amino acid position 531 of the rpoB gene, or a katG gene, and the sequences of the probes differ at amino acid position 315 of the katG gene. For Influenza virus, preferably the target sequence comprises a segment of a gene or genome that confers drug resistance to the organism, and the gene is a protective antigen gene that confers drug resistance to baloxavir marboxil wherein the probes differ at amino acid position 38 of the protective antigen gene. Or the gene is a neuraminidase gene that confers drug resistance to oseltamivir, and wherein the neuraminidase gene is an N1 gene and the probes differ at amino acid position 275 of the N1 gene, and wherein the neuraminidase gene is an N2 gene and the probes differ at amino acid position 292 of the N2 gene. Preferably the conserved region is about 100 to about 300 nucleotides in length. Wherein the pair of nucleic acid primers each have a GC content of about 65%, preferably the mixture contains a reducing agent such as, for example, DMSO or TCEP at a concentration of from about 0.01 mM to about 500 Mm, or preferably the concentration is from about 1.0 mM to about 50 mM.

Preferably the probes are differentially labeled with a fluorochrome such as, for example, FAM, JOE, ROX, VIC, ABY, JUN, TAMRA, NED, TET, HEX, PET, or a combination thereof. Preferably the PCR is qPCR with a temperature cycling that provides for denaturation followed by annealing and extension, and further comprises positive and/or negative controls. Preferably temperature cycling comprises multiple cycles of from about 15° C. to about 25° C. followed by from about 50° C. to about 80° C. Preferably one probe comprises a wild type target sequence and one probe comprises the mutated sequence or SNP. Preferably the label detected predominantly is the label associated with the 5-terminus of the one probe, the label detected predominantly is the label associated with the 5-terminus of the other probe, or the labels associated with the 5-terminus of each probe are detected in substantially equal quantities. Labels detected may be predominantly those label associated with the 5′-terminus of each probe or labels detected are predominantly the labels associated with the 3′-terminus of each probe. Preferably, the method is performed within 8 hours or less, within 4 hours or less, or within 2 hours or less.

Preferably the method detects genetic variations such as an allele associated with a genetic disease or disorder in a mammal. Preferably, the mutations conferring drug resistance are mutations conferring resistance to an antibiotic or a chemotherapy. Preferably the genetic disease or disorder comprises expression or absence of expression of an enzyme, immune system functioning, generation of a B cell or T cell response to an infection, or resistance or sensitivity to a drug. Preferably the method is performed simultaneously on multiple different mixtures, such as, for example, the organism is a Mycobacterium and the method detects multiple genetic variations of the same organism having with multiple drug resistances, or of different organisms each with a different drug resistance profile.

Preferably the method is indicative of the presence of a pathogen or the presence of cancerous tissue, wherein the pathogen comprises one or more of a virus, a bacterium, a fungus or a parasite. Preferably the virus is one or more of a DNA virus, an RNA virus, a positive or negative single-strand virus, a double strand virus, an orthomyxovirus, a paramyxovirus, a retrovirus, a flavivirus, a filovirus, a lentivirus, an influenza virus, a human immunodeficiency virus, a hepatitis virus, or an ebola virus. Preferably the bacterium is Mycobacterium tuberculosis, Klebsiella sp., Clostridium sp., Escherichia sp. (e.g., E. coli), Pseudomonas sp. (e.g., P. aeruginosa), Neisseria sp. (N. gonorrhoeae), Francisella tularensis, Yersinia pestis, or Vibrio cholera. Preferably the parasite is Plasmodium sp. (e.g., P. falciparium; malaria), a nematoad, Toxoplasma sp. (e.g., T. gondii), Preferably the genome is obtained from bodily fluid and/or tissue of the patient. Preferably the biological sample is provided in a molecular transport medium and the molecular transport medium contains a chaotrope, a detergent, a reducing agent, a chelator, a buffer, and an alcohol, together present in an amount sufficient to lyse cells, denature proteins, inactivate nucleases, kill pathogens, and not degrade nucleic acid. Preferably, the quantitative polymerase chain reaction is carried out in an aqueous mix comprising: a polymerase and optionally a reverse transcriptase; a mix of deoxynucleotide triphosphates comprising about equivalent amounts of dATP, dCTP, dGTP and dTTP, a chelating agent, an osmolarity agent, an albumin, a magnesium salt; and a buffer.

Another embodiment of the invention is directed to methods of treating a disease or disorder caused by the at least one microorganism strain or serotype with the antimicrobial compound identified by the methods of the invention. Preferably, treatment comprises the targeted killing of the specific pathogen that is the causative agent of the disease or disorder by a therapy determined from the information obtained from the method disclosed herein. Preferably, the information determined also identifies the effective therapeutic dose.

Another embodiment of the invention comprises kits containing reagent vessels preferably including one or more of chemical reagents, primers and polymerases for PCR sequencing and analysis according to the methods disclosed herein. The sample to be analyzed is mixed with a reagent vessel that preferably contains chemical components sufficient to kill all pathogens present in the sample, inactivate nucleases in the sample, and maintain the integrity of the nucleic acids and fidelity of sequences rendering the sample safe for transportation and subsequent manipulation (e.g., PrimeStore™). Kits contain multiple primer pairs that target specific sequences of a particular organism that are known to be related to drug resistance. Extracted nucleic acid is preferably combined with another chemical reagent composition such as, for example PrimeMix™ that facilitates nucleic acid testing such as, for example, qPCR analysis. Kits may contain positive control sequences, negative control sequences and/or sequences that specifically hybridize (under the desired high or low stringency hybridization conditions) to a particular target sequences that is characteristic for the presence of a pathogen.

Other embodiments and advantages of the invention are set forth in part in the description, which follows, and in part, may be obvious from this description, or may be learned from the practice of the invention.

### DESCRIPTION OF THE FIGURES

FIG. 1 Evaluation of KatG isoniazid resistance by qPCR with HN878 MTB.

FIG. 2 Evaluation of KatG isoniazid resistance by qPCR with mixed-strain.

FIG. 3 Evaluation of 16 patient isolates.

### DESCRIPTION OF THE INVENTION

Rapid analysis of gene sequences associated with allelic variations such as drug resistance is a major challenge for successful treatment of many diseases and disorders. Conventional techniques require culturing of biological material suspected of containing an infectious agent and testing of various compounds requiring highly trained personnel and costly equipment. Moreover, the time required for testing delays the initiation of effective treatment of the patient.

A qPCR protocol for rapid characterization of resistance gene sequences has been surprisingly discovered that provides both accuracy and speed, and with minimal cost, allowing for proper patient treatment to be initiated almost immediately. The invention comprises a protocol for analysis of allelic variations of nucleic acid within cells such as mammalian cells, and/or infectious microorganisms, such as Mycobacteria and Influenza virus, which includes the simultaneous detection of multiple variations with a single biological sample. Improved and faster MTB detection of drug resistance is critical for low resource areas. The method applies to genetic detection of genomic regions in Mycobacterium tuberculosis known to confer antibiotic resistance. Such regions include, but are not limited to genetic loci that confer resistance to rifampin, isoniazid, pyrazinamide, fluoroquinolones, delamanid, bdeaquiline, and linezolid.

In particular, the method of the invention provides a one-step analysis allelic variation to determine enzyme activity with a sample or mammalian cells, drug resistance within the genome of bacteria, virus, fungi or parasites, and the nucleic acids of a biological sample obtained from a patient suspected of having a disease or disorder. Variations detected include those associated with drug resistance and/or sensitivity attributable to the genome of the microorganism, the presence and/or absence of enzyme activity of mammalian cells, single nucleotide polymorphisms (SNPs), nucleic acid markers associated with diseases and disorders, multiple drug resistance alleles, and also variations attributable to multiple different causes. Preferably, the method identifies one or more nucleic acid variations, which may be attributed to infected cells and/or the infectious microorganisms, associated with drug resistance in a biological sample containing an infectious agent.

This rapid, standardized, cost-effective protocol allows for the identification of mutations that possess one or more alterations of a DNA, RNA, protein and/or peptide sequence. For sample sequences that are RNA, the RNA sequence of interest in the sample is typically reverse transcribed to DNA for PCR analysis. Preferably identified and characterized are one or more gene mutations that provide a microorganism with resistance to an antibiotic. Preferred mutations that are identified with the methods of the invention are located in one or more sites within an amino acid coding region, a transcription promoter or termination site, a stop or start codon, a site within a non-coding region, a splice junction site, a modification site, a transcription or translation factor binding or recognition site, one or more sites that contribute to a three dimensional structure, or a combination thereof, Preferred genes that are analyzed include MTB genes associated with first and second-line MTB drug resistance. Preferred examples of MTB-associated genes include, for example, rpoB (rifampin), katG and inhA (isoniazid), gyrA and gyrB (fluoroquinolones), pncA and panD (PZA or pyrazinamide) and rrs(16s) (aminoglycosides, amikacin, kanamycin, capreomycin, streptomycin) and rspL (streptomycin) (preferred genes for influenza antivirals). Preferred genes for influenza antivirals: oseltamivir-neuraminadase and baloxivir-polymerse acidic protein. The method can be designed to a targeted sequence that indicates sensitivity or resistance to antiviral drugs used for influenza treatment, and antibacterial agents used for treating bacterial infections such as MTB, particularly including multidrug resistant (MDR) or extensively drug resistant (XDR) strains.

Preferably, the invention is directed to obtaining a biological sample from a patient. Sample materials may be liquid, solid or mixed liquid and solid and preferably include, for example, blood samples, tissue sample, mixed tissue samples, saliva, throat swabs, biopsied tissue and combinations thereof. Samples are preferably collected in a molecular transport medium such as, for example, PRIMESTORE™ (Longhorn Vaccines and Diagnostics, LLC; Bethesda, Md.) that provides immediate sterilization, allows for safe handling and non-refrigerated worldwide transport, and maintains the integrity of nucleic acids within the sample for subsequent nucleic acid testing. Preferably, the nucleic acids of the sample contained within the molecular transport medium do not lose structural integrity or sequence fidelity after storage in the molecular transport medium for days, weeks, months, or longer.

Nucleic acid material is extracted from the biological sample for subsequent polymerase chain reaction (PCR), to which is added nucleic acid primers pairs, two nucleic acid probes, each containing substantially the same target sequence of interest, and a mixture of ingredients for the PCR reaction.

Primer pairs are selected with sequences that span the target sequence of interest, which is preferably, a conserved region of the nucleic acid, and contains the suspected allelic variation. The target sequence of interest and/or the amplicons produced are preferably from about 50 to about 1,000 nucleotides in length, preferably from about 100 to about 500 nucleotides in length, preferably from about 100 to about 400 nucleotides in length, preferably from about 100 to about 300 nucleotides in length, and more preferably from about 100 to about 200 nucleotides in length.

The two nucleic acid probes each contain substantially the same sequence of the target sequence of interest, but differ in five or less nucleotides of the target sequence, preferable four or less nucleotides, preferable three or less nucleotides, preferable two or less nucleotides, and preferable one nucleotide. Probes sequences may substantially overlap with each other, overlap only of the target sequence, or be otherwise identical except for the differing nucleotide(s) of the target sequence. Probes sequences preferably hybridize to the target sequence although with different hybridization strengths and are preferably smaller than the size of amplicons to be generated by PCR. Preferred probe pairs contain the allelic variation that comprises a SNP, a genetic abnormality, or a mutation. Preferably the probes contain a sequence that imparts resistance to an antibiotic or therapeutic compound, such that one probe contains the wild-type sequence and the other probe contains the mutated sequence, wherein either the wild-type or the mutation is attributable to resistance.

Probes are differentially labeled at their respective 5-terminus, 3′terminus, and/or both. Labels include any identifiable tag that provides a detectable signal such as, for example, a color, a dye, ionizing or non-ionizing radiation, a resonance, an electrical signal, enzyme activity or a combination thereof. Preferably, the label does not sterically hinder probe hybridization to the target sequence. Dyes include, for example, modified nucleotides or proteins, chemical moieties, nucleic acid or protein dyes, conjugated dyes, fluorescent chromophores, and/or combinations thereof. Preferred fluorescent chromophores include, for example, fluorophores derivatives of rhodamine (TRITC), coumarin, and cyanine, also FAM (carboxyfluorescein), JOE, ROX, VIC, ABY, JUN, TAMRA, NED, TET, HEX, PET, and combinations thereof.

The mixture of ingredients for the PCR reaction preferably includes a buffer, salts including a magnesium salt, a heat-stable polymerase (e.g., Taq polymerase), a mixture of deoxynucleotide triphosphates (e.g., dNTPs including approximately equivalent amounts of dATP, dTTP, dCTP, dGTP), and nuclease-free water. Preferably the mixture comprises PRIMEMIX™, a commercially available PCR-ready mixture (Longhorn Vaccines and Diagnostics, LLC; Bethesda, Md.). PCR may also involve RT-PCT (reverse transcriptase-PCR), whereby the target sequence comprises RNA, which is transcribed to DNA for nucleic acid testing.

In conditions where the GC content of the sequence of interest is high (about 65% or greater), PCR assay sensitivity is particularly challenging due to the high denaturation temperatures required. In such cases, it was surprisingly discovered that a PCR buffer containing a nonconventional reducing agent overcomes this limitation. Reducing agents include, for example, 2-mercaptoethanol ((3-ME), tris(2-carboxyethyl) phosphine (TCEP), dithiothreitol (DTT), formamide, dimethylsulfoxide (DMSO), or any combination thereof. Preferred concentration of the reducing agent in the PCR mixture is from about 0.01 mM to about 500 mM, more preferably from about 1.0 mM to about 50 mM, and even more preferably TCEP is present at a final concentration of about 4.5 mM.

PCR is carried out according to standard protocols or modified to accommodate the particular sample nucleic acids, primers, and/or probes. As all sequences are known or easily determined, those of ordinary skill in the art can determine the appropriate thermocycling temperatures and times desired for the allelic variation to be detected. Standard PCR conditions comprise about 20 to about 40 cycles of denaturation and annealing followed by primer elongation, and preferably 30 cycles. Temperature cycling comprises denaturation at from about 70° C. to about 98° C., for about 20 seconds to about 50 seconds, with annealing at from about 40° C. to about 65° C. for about 20 second to about one minute, followed by elongation at about 60° C. to about 80° C. for about 30 second to about five minutes or longer. Modification of standard PCR comprises more or less cycling, and greater or lesser temperatures, as may be appropriate as determined by those skilled in the art, for the particular sample nucleic acid, primers, and/or probes, for denaturation, annealing, and elongation as desired. Preferably, PCR of the invention comprises denaturation followed by an almost simultaneous annealing and elongation.

Quantitative polymerase chain reaction (qPCR) is preferred such that the amount of each label can be determined. Just as PCR, the qPCR technique is used to amplify a segment of DNA, i.e., an amplicon. Preferably, qPCR comprises the use of internal, fluorescently labeled primers that can be detected at the end of each successive cycle by a fluorometer in real-time. Real-time PCR instruments do not require the visualization of PCR amplicons using tedious gel electrophoresis approaches, and is quite rapid in comparison to NGS (e.g., 1-2 hours for qPCR vs. 4-6 hours using standard PCR and gel-based visualization). Furthermore, the sensitivity of qPCR is superior to conventional PCR due at least in part to highly sensitive detection limits in the real-time fluorometer in contrast to standard visualization of amplicons using ethidium bromide or other intercalating dyes to visualize PCR amplicon products. Thus, a qPCR approach for disease detection is of particular advantage over culturing methods. Furthermore, qPCR is advantageous in the developing world since it can be employed as near to the patient as possible. Following PCR, the labels are detected, preferable quantitatively or relatively, or compared to a baseline reading, and the presence and preferable quantity of each label determined.

During PCR amplification, one or the other probe will bind preferably to the target sequence. The probe that most strongly anneals at the temperature selected will be cleaved during thermocycling and the label attributable to that probe can be detected. For probes labeled, for example, with fluorochromes, the cleaved probe will emit fluorescence that is detected by the real-time fluorometer and the uncleaved probe with remain quenched and not emit fluorescence. Because the label for each probe is different, the probe which most strongly hybridizes is easily determined. When that probe contains the wild-type sequence, the target sequence of the genome being analyzed is determined to be wild-type. When the cleaved probe contains a mutated sequence, the target sequence of the genome being analyzed is determined to be the mutant sequence.

For labels that remain detectable before and after cleavage, the cleaved and uncleaved probes, if needed, can be separated by chromatography, by electrophoresis, by mass spectrometry, by affinity, by filtration, by centrifugation (e.g., spin columns), or by combinations or other methods well known to those skilled in the art, and the relative amount of each label determined.

In addition, quantitative assessment will also allow determination of the presence of multiple different allelic variations either in the same organism or when nucleic acids from multiple organisms are present and tested. For example, where a percentage of the variations are wild-type and another percentage are mutants, the amount of each label detected will reflect the percentages of each.

By analyzing the allelic variance determined by the methods as disclosed herein, an effective drug therapy can be selected from immediate treatment of a patient. This provides a significant advancement over conventional analysis that requires culture of the diseased cells and exposure to the various drug therapies to identify the effective treatment and preferably, the effective dose as well. Thus, after a single PCR analysis, the effective treatment can begin. Preferably the analysis is performed in 6 hours or less, more preferably 4 hours or less, and more preferably 2 hours or less.

The methods disclosed herein allow for detection of a genetic variation within a targeted sequence amongst a population of sequences that may be: 1) homogeneous for the mutation of interest, 2) homogeneous for a sequence that does not contain the mutation (generally referred to as wild-type sequence), and 3) a population of sequences comprising the wild type and mutant sequences. This method may be used to detect any region of any genome comprising an organism containing DNA or RNA as the genetic material including, for example, gene that are eukaryotic, prokaryotic, or fungal in nature. In preferred example, the method is used to detect a mutation in DNA or RNA from a disease causing organism or microbe such as, for example, a microbe associated with a viral, bacterial, or fungal infection.

Disease-causing organisms that can be evaluated according to the method disclosed herein include different strains of bacteria, virus, fungus, and parasites, or combination thereof. Exemplary organisms include, but are not limited to DNA virus, an RNA virus, a positive or negative single-strand virus, a double strand virus, orthomyxovirus, paramyxovirus, Morbillivirus (e.g., Rubeola), retrovirus, flavivirus, filovirus (e.g., Ebola, Marburg), lentivirus, hanta virus, herpes virus (e.g., VZV, HSV I, HSV II, EBV), hepatitis virus (e.g., A, B, C, non-A, non-B), Arbovirus (e.g., Zika virus), Dengue virus, Lassa virus, Hantavirus, Influenza virus (e.g., H5N1, H1N1, H2N5, H7N9), Respiratory Syncytial Virus, HIV, or Ebola virus. Exemplary organisms also include but are not limited to Salmonella sp., Staphylococcus sp., Streptococcus sp., Mycobacteria (e.g., M. tuberculosis, M. leprae, M. smegmatis, M. bovis), Bacillus anthracis, Plasmodium (e.g., Plasmodium falciparum), Shistosomiasis (e.g., Schistosoma mansoni), Francisella tularensis, Clostridium difficile, Meningococcal infections, Pseudomonas infections, Yersinia pestis, and Vibrio cholerae. Preferably, methods of the invention are of particular use for detecting sequences of influenza virus and Mycobacterium tuberculosis (MTB), that reveal drug resistance or sensitivity.

The invention is also directed to the detection of allelic variation associated with cancer or cell malignancy, which also can be used to determine drug or therapeutic sensitivities and resistances, on mammalian cells to determine the expression and/or the level of expression of certain genes.

The following examples illustrate embodiments of the invention, but should not be viewed as limiting the scope of the invention.

### Example 1

The ability to improve MTB detection with sensitive real-time PCR and then rapidly qPCR resistance genes is critical especially for low resource areas. Since PS-MTM rapidly kills MTB and preserves the DNA at ambient temperature and above, specimens can be efficiently transported for real-time PCR and sequencing to improve detection of drug resistant strains and optimize patient therapy. Previous studies have shown the benefit of sequencing MDR strains from patients who have come to the US from countries with MDR and XDR to identify qPCR assay resistance mutations. An additional advantage of provides the ability to detect more than one population, such as, for example, heteroresistance in the patient's specimen. Heteroresistant characterization is important for patient care, especially if MTB subpopulations that are resistant to key antibiotics as these become the predominant patient strain. This example also demonstrates the feasibility of transporting sputum specimens efficiently to central and regional labs to provide support to rural clinics. Understanding the epidemiology and the role of mobile populations in rapidly changing resistance patterns, particularly in rural African settings is important to treat and eradicate TB. Without adding extra training staff or infrastructure, patient sputum specimens from rural areas can be transported to labs with highly trained personnel and state of the art qPCR equipment to support MTB patient care surveillance and research.

Characterization of drug resistance genes of MTB is critical for the appropriate treatment of tuberculosis (TB). Molecular detection and new assays are rapidly providing new tools to diagnose and improve treatment of drug resistant TB.

As shown in Table 1, qPCR was used to characterize MTB rpoB and katG drug resistance genes directly from specimens collected and transported at ambient temperature from South Africa to the United States in PrimeStore® MTM (PS-MTM).

TABLE 1
Clinical Isolate Tests

| | | PrimeMixTB | PrimeMixTB | PrimeMix |
|---|---|---|---|---|
| | Mi-Seq katG | Ct value | Ct Value | katG |
| No. | Isoniazid | (6110) | (1081) | DST |
| 1 | S-315-T | 23.9 | 25.5 | Resistant |
| 2 | R-463-L | 22.2 | 24.7 | Wild Type |
| 3 | R-463-L | 22.2 | 24.3 | Wild Type |
| 4 | S-315-T | 28.7 | 30.1 | Resistant |
| 5 | R-463-L | 23.1 | 24.6 | Wild Type |
| 6 | R-463-L | 25.8 | 28.8 | Wild Type |
| 7 | S-315-T/ | 26.2 | 28.2 | Resistant |
| | R-463-L | | | |
| 8 | S-315-T/ | 23.1 | 25.8 | Resistant |
| | R-463-L | | | |
| 9 | S-315-T | 27.7 | 28.7 | Resistant |
| 10 | R-463-L | 21.6 | 24.3 | Wild Type |
| 11 | S-315-T/ | 26.1 | 27.8 | Resistant |
| | D-448-A | | | |
| 12 | S-315-T | 25.0 | 25.2 | Resistant |
| 13 | S-315-T | 22.4 | 23.2 | Resistant |
| 14 | S-315-T | 27.8 | 28.5 | Resistant |
| 15 | S-315-T | 27.2 | 27.3 | Resistant |
| 16 | S-315-T | 22.6 | 24.8 | Resistant |

Shows ~100% concordance with NGS results of actual sequencing

These genes confer resistance to first line drugs, rifampicin and isoniazide, respectively. The significant of this work is not simply detecting drug resistance, but the spend at which resistance can be determined and, thus, treatment can begin. One important element is obtaining and maintaining high quality DNA from biological specimens. This was accomplished by collecting and transporting specimens in PRIMESTORE, a well-known and well-regarded molecular transport medium that enables rapid, centralized high throughput processing of specimens.

A set of oligonucleotide primers that target a specific region of a genome was selected. Primers represent orientations in the forward and reverse direction such that they amplify a specific region within a gene of interest. The regions selected for the forward and reverse primer sequences are selected to ensure that the primers are designed from conserved areas and, preferably, that do not contain variant sequences. Each primer was labeled at the 5′ end with a specific fluorochrome that when hydrolyzed during successive PCR cycles, release a signal at a characteristic wavelength. Sequences of bacteria collected from varying geographies, niches, time periods, and hosts can be used to assess the level of homogeneity in the target primer sequences.

Binding within the region defined by the forward and reverse primers is determined by two probe sequences. The probes are homologous with the exception of one nucleotide (e.g., A, T, C, G), and may be partially overlapping, or non-overlapping in comparison to each other. In the presence of the wild-type sequence, the probe with the wild-type sequence binds and prevents elongation of one primer and not the other. In the presence of the mutant sequence, the probe with the mutant sequence binds and prevents elongation of the other primer.

The labeled primers and two probes were included in a molecular transport medium, PrimeMix™ and a qPCR assay performed. The nature of the assay enables genotyping a single nucleotide polymorphism (SNP). The method may employ a 5′ nuclease assay for amplifying and detecting a specific target. Since there are two probes types present, the probe with optimal specificity will bind, and such the primer that would otherwise anneal and elongate that sequence will be prevented and subsequently cleaved during thermocycling. The cleavage event is detected by the real-time fluorometer, whereas the other probe remains uncleaved and the fluorescent signal quenched. Therefore, two probes in a single assay enable the detection of a specific SNP.

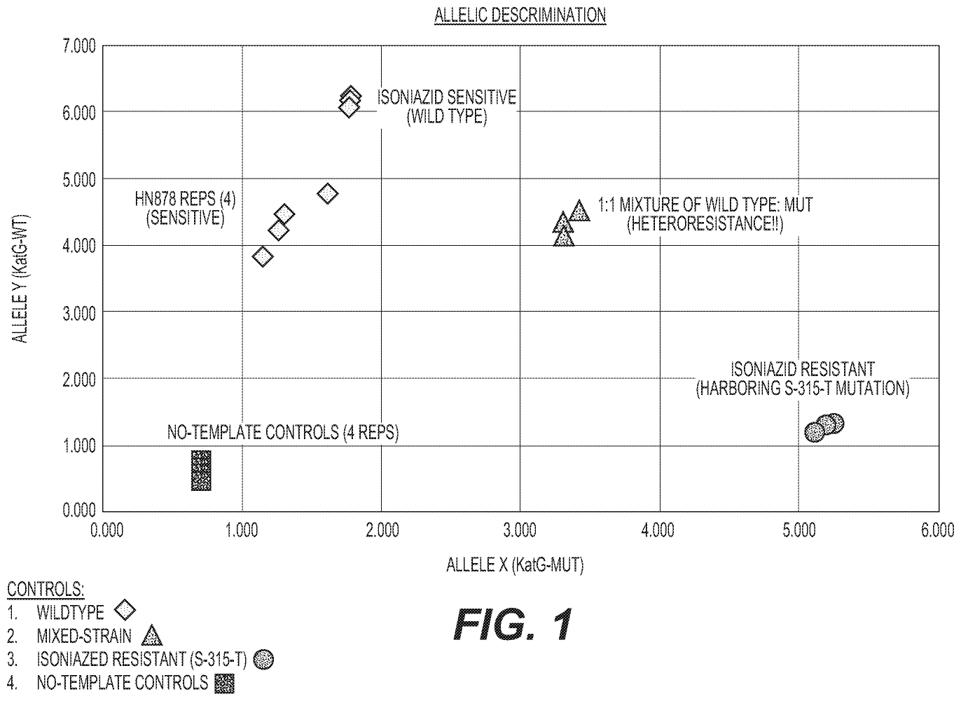

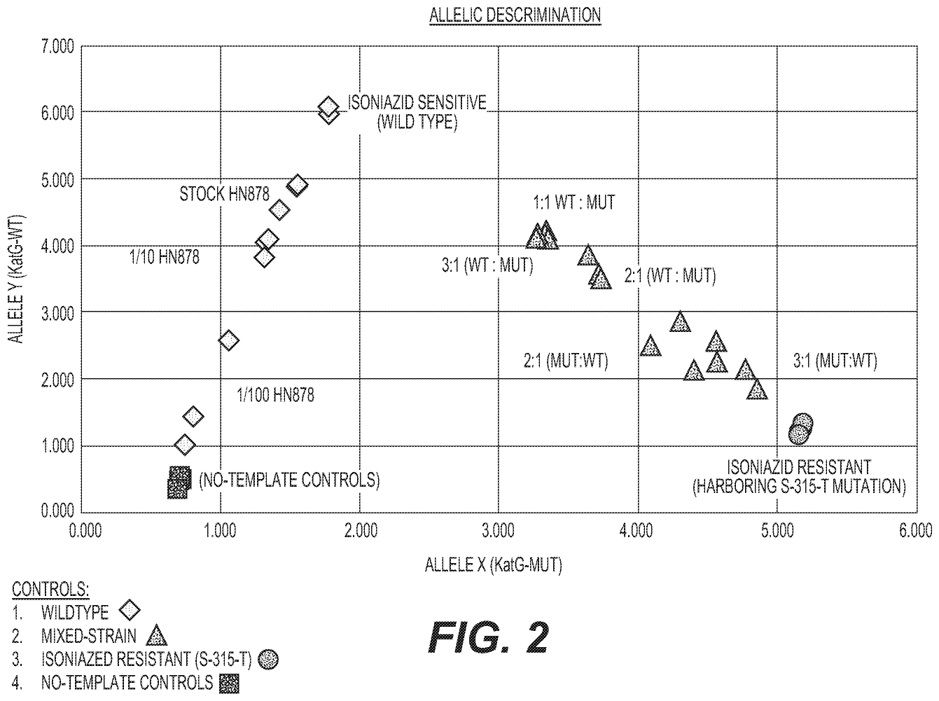

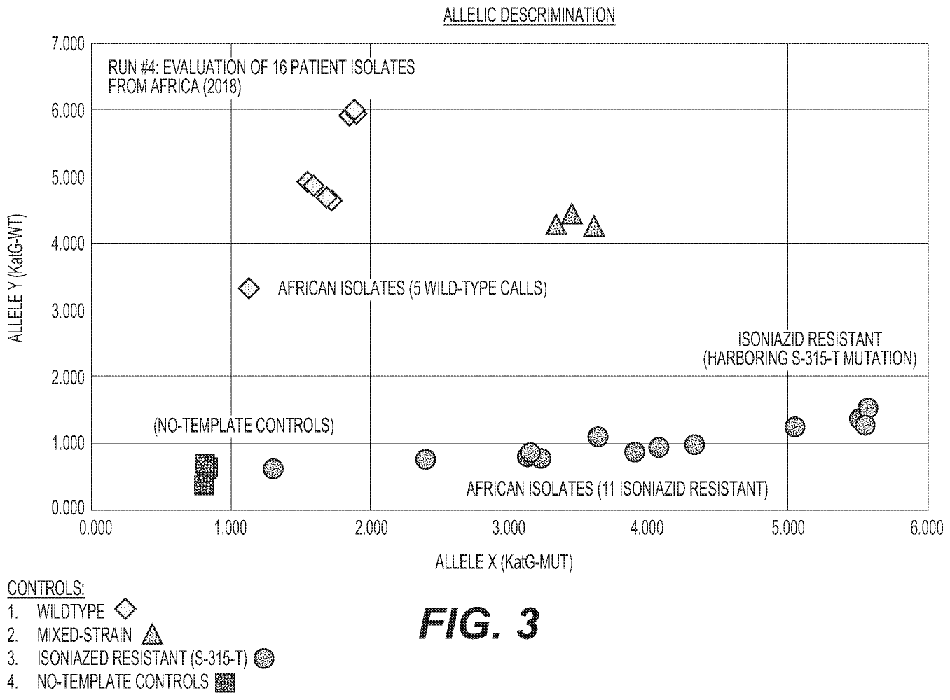

As shown in FIGS. 1-3, this method was used to detect genetic variations in 16 clinical isolates form patients infected with Mycobacterium tuberculosis (MTB). The allelic variations were known to confer resistance to specific antibiotics. The drug sensitivities of each isolate were not previously known, and all were subsequently sequenced by next generation sequencing (NGS) to determine exact allelic variations. As demonstrated in these Figures, not only were both sensitive and resistant strains identified, but also mixed strains showing hetero-resistance. This method represents a substantial improvement over current technologies for determining antibiotic resistance in MTB. The most widespread method for determining antibiotic resistance is by cell culture analysis. The standard for identification of drug resistant TB strains is culture-based drug susceptibility testing (DST) often performed using an automated BACTEC™ MGIT™ 960 system (Becton Dickinson, Silver Sparks NV, USA). However, culture-based DST is time-consuming (e.g., weeks to months), requires propagation of potentially infectious strains, and is cost prohibitive particularly in resource limited countries. Furthermore, automated culturing requires trained personnel, and regular and expensive instrument maintenance. In contrast, drug resistance testing via genetic analysis using PCR (e.g., qPCR) enables rapid determination with minimal sample preparation, minimal equipment, and expense. The Illumina MiSeq platform is capable of sequencing up to 24 whole MTB genomes per run with an average reproducible coverage depth of about 30 times. qPCR instruments require less maintenance and training, have been utilized for a decade or more, and available worldwide.

As shown in FIG. 5, two MTB drug resistance assays were designed to rapidly detect mutations known to confer resistance to rifampin and isoniazid, two important antibiotics used in the treatment multi-drug resistant tuberculosis (MDR-TB). For rifampin, the most prevalent mutation (S-531-L) was targeted in the rpoB gene. For isoniazid, a region of the katG gene comprising the S-315-T mutation, known to confer resistance to isoniazid was exploited.

In this example, the primer set is specific to amplification of a 100-200 base pair region of the katG gene was optimized and developed for sensitive and specific PCR spanning the S-315-T mutation. The primer melting temperatures are known or easily determined by one skilled in the art and a Tm determined for thermocycling parameters that promote annealing and extension PCR steps to be incorporated into a single temperature and specified duration. Thus, thermocycling is not a typical three-step process that includes: 1) denaturation, 2) annealing, and 3) extension, but rather a 2-step approach where thermocycling is simply denaturation, and subsequent annealing/extension combined.

Internal to the primer pair are probe sequences that differ by a single nucleotide. One probe is reflective of the wildtype sequence and the other of the mutational sequence. Each primer is labeled with a fluorochrome that emits fluorescence at a characteristic wavelength. Fluorochromes include, for example, FAM, VIC, TAMRA, NED, or a combination thereof.

PCR amplification cycling is carried out with the selected primers and probes. The probe, either the wildtype or mutational probe, preferably binds to the target sequence depending on the population of target sequences in the sample well. For the sequence conferring resistance is present in the sample, then the mutant probe will anneal in preference to the wild type probe. The probe that most strongly anneals will be cleaved during thermocycling and the respective fluorochrome emits fluorescence that is detected by a real-time fluorometer, whereas the flourochrome of the non-annealed probe is quenched. Fluorescence is greater in comparison to initial baseline readings of each fluorochrome before run initiation. An endpoint difference in fluorescence is detected within 2 hours or less. This method showed that resistance or sensitivity to the antibiotic isoniazid was determined as it pertains to MTB infections within a human host, and represents a significant improvement over traditional culturing for determining isoniazid resistance which can take weeks to months.

The amplification determined at the endpoint using synthetic targets for: 1) wild type, 2) mutant, and 3) 1:1 ratios of mutant and wildtype sequences are determined. Both positive and negative controls were included HN878 MTB, which were shown to be wild type, and thus isoniazid sensitive (see FIGS. 1-3).

### Example 2

qPCR was performed on the 16 clinical isolates testing for resistance or sensitivity to (i) rifampicin, (ii) isoniazid, (iii) fluoroquinolones, (iv) aminoglycosides, (v) pyrazinamide. Results are shown in Table 2.

TABLE 2
Genetic Characterization of Drug Resistance Signatures in 16 Clinical Isolates from Africa

| | | Prime Seq TGS | | | | PrimeMix MTB | PrimeMix MDR | |
|---|---|---|---|---|---|---|---|---|
| No. | Isolate | rpoB | katG | gyrA | pncA | 6110 | isoniazid | rifampin |
| | | | | E21Q | | | katG(S315T) | rpoB(S531L) |
| LH-1 | DR93RS | Q513K | S315T | D94A | H51Q | 23.9 | T | S |
| | | | | S95T | | | | |
| LH-2 | DR98 | S53IL | R463L | A90VA | MIT | 22.2 | S | L |
| | | | | S95T | | | | |
| LH-3 | DR105 | S53IL | R463L | A90V | Y103H/Y | 22.2 | S | L |
| | | | | S95T | | | | |
| LH-4 | DR111 | NA | S315T | A90V | V21del | 28.7 | T | L |
| | | | | S95T | | | | |
| LH-5 | DR113 | S53IL | WT | D94G | WT | 23.1 | S | L |
| LH-6 | DR115 | S53IL | R463L | D94GA | WT | 25.8 | S | S |
| | | | | S95T | | | | |
| LH-7 | DR129 | D516V | S315T | D94G | 173FS | 26.2 | T | S |
| | | | R463L | S95T | | | | |
| LH-8 | DR133 | D516V | S315T | S95T | 173FS | 23.1 | T | L |
| | | | R463L | | | | | |
| LH-9 | DR1825 | S53IL | S315T | A90V | V21del | 27.7 | T | L |
| | | | | S95T | | | | |
| LH-10 | DR160s | S53IL | R463L | A90V | Y103H | 21.6 | S | L |
| | | | | S95T | | | | |
| LH-11 | DR119R | S53IL | S315T | | S59P | 26.1 | T | L |
| | | | D448A | | | | | |
| LH-12 | DR107 | S53IL | S315T | S95T | V139A | 25 | T | L |
| LH-13 | DR1535 | S53IL | S315T | S95T | WT | 22.4 | T | L |
| LH-14 | DR100 | S53IL | S315T | S95T | WT | 27.8 | T | L |
| LH-15 | DR89 | S53IL | S315T | S95T | WT | 27.2 | T | L |
| LH-16 | DR1245 | D516V | S315T | D94G | L151S | 22.6 | T | S |

WT = wild type compared to MTB H37Rv;
BJ = Beijing-like Dengue;
rpo = rifampicin;
katG = isoniazid;
inhA = isoniazid;
gryA = fluoroquinolones;
pncA = pyrazinamide;
Underlined = Resistance conferring;
V21del = deletion of T;
172FS = insertion of G GAG to GGAG

### Example 3

In recent years, next-generation sequences (NGS) has emerged as the method of choice for genetic characterization of Mycobacterium tuberculosis (MTB) mutations that confer multi-drug resistance (MDR). However, NGS is cost prohibitive, and requires laborious sample preparation, a highly trained biological and bioinformatics staff, and a high complexity laboratory. We developed a simplified, rapid qPCR allelic discrimination method for detection of high prevalence mutations in the rpoB and katG genes, which confer resistance to rifampin and isoniazid, respectively. These assays were evaluated using 16 phenotypically multi-drug resistant (MDR) South African clinical isolates and compared to mutations identified by NGS.

Two rpoB assays targeting mutations S-450-L and D-435-L, and a katG assay specific for S-315-T were designed and optimized using targeted DNA controls on an ABI-7500 instrument. Following initial optimizations, a total of 0.1 mL MGIT culture from each of the 16 isolates was transferred into tubes containing 1.5 mL of PrimeStore® and shipped at ambient temperature from Pretoria to San Antonio, Tex. for analysis. Mutations detected by qPCR were compared to those obtained by MiSeq NGS.

Using these rpoB assay targets, resistance-conferring mutations were identified in 15 of 16 isolates (94%; 12 (75%) were S-450-L and 3 (19%) were D-435-L). In these 15, NGS confirmed the results obtained by qPCR. The single isolate testing ‘wildtype’ by qPCR was shown by NGS to harbor a rpoB Q-423-K mutation not covered by these assay targets. There were 11 isoniazid resistant isolates (69%) identified by NGS harboring a S-315-T mutation in the katG gene that were also detected using the katG qPCR assay.

These data indicate that this rapid qPCR approach accurately detected rifampin and isoniazid resistance in patient isolates (see Table 3). One isolate identified by NGS contained a Q-423-K substitution in the rpoB gene, a less prevalent but documented rifampin resistance-conferring mutation. A Q-423-K target assay and other less prevalent rifampin and isoniazid resistance qPCR assays are currently being developed. Rapid qPCR detection of MDR-TB is a promising approach, particularly in low-resource areas where classical culture DST or complex NGS methods are not available.

TABLE 3
Detection of rifampin and isoniazid resistance-conferring mutations
by qPCR in 16 clinical isolates from Africa compared to NGS.

| | | | qPCR Detection | | |
|---|---|---|---|---|---|
| | | | Rifampin | | Isoniazid |
| MiSeq NGS | | | resistance | | resistance |
| | rpoB | katG | rpoB | rpoB | katG |
| Isolate | (rifampicin) | (isoniazid) | (S-450-L) | (D-435-V) | (S-315-T) |
| LH-1 | Q-513-K | S-315-T | S | D | T |
| LH-2 | S-531-L | R-463-L | L | D | S |
| LH-3 | S-531-L | R-463-L | L | D | S |
| LH-4 | S-531-L | S-315-T | L | D | T |
| LH-5 | S-531-L | WT | L | D | S |
| LH-6 | S-531-L | R-463-L | L | D | S |
| LH-7 | D-516-V | S-315-T, | S | V | T |
| | | R-463-L | | | |
| LH-8 | D-516-V | S-315-T, | S | V | T |
| | | R-463-L | | | |
| LH-9 | S-531-L | S-315-T | L | D | T |
| LH-10 | S-531-L | R-463-L | L | D | S |
| LH-11 | S-531-L | S-315-T, | L | D | T |
| | | D-448-A | | | |
| LH-12 | S-531-L | S-315-T | L | D | T |
| LH-13 | S-531-L | S-315-T | L | D | T |
| LH-14 | S-531-L | S-315-T | L | D | T |
| LH-15 | S-531-L | S-315-T | L | D | T |
| LH-16 | D-516-V | S-315-T | S | V | T |

WT = Wild type compared to M. tuberculosis H37Rv. S = Serine; T = Threonine; D = Aspartic Acid; V = Valine; L = Leucine. BOLD = Resistance conferring mutation.

### Example 4

Poor-quality cough specimens (n=61) from presumptive tuberculosis cases were cultured and GeneXpert MTB/RIF (Xpert) successfully performed on samples transferred by flocked swab into PrimeStore molecular transport medium (PS-MTM). M. tuberculosis was grown in culture from 13 (21.3%) and Xpert reported 15 (24.2%) positive, of which 10 concordant. RT-PCR of PS-MTM samples showed enhanced sensitivity; three positives were missed by Xpert, five by culture and 3 more detected for a total of 21 positives (34.4%).

Clinical specimens unfit for laboratory processing represent missed opportunities for diagnosing tuberculosis. In a recent report on global tuberculosis (TB) rates, the World Health Organization expressed concern about the under-reporting of active cases of TB. Compared to estimated rates, approximately 30% of cases go undetected annually. A contributing factor to under-diagnosis of active TB cases is the rejection of specimens for GeneXpert MTB/RIF (Xpert) processing on the basis of low volume or poor quality. In South Africa, cough specimens with volume less than 5 ml are considered of poor-quality (e.g. bloody, salivary and samples with food particles) and not eligible for analysis. An objective of this experiment is to show that samples not meeting routine processing requirements can be optimized for detection of Mycobacterium tuberculosis.

This experiment is designed to show, among other aspects, that small quantities of sputum samples transferred to molecular transport medium (MTM) before processing in most PCR based assay platforms, including real-time PCR and Xpert, can provide for detection of additional cases that might otherwise be missed in conventional approaches. PrimeStore-MTM (PS-MTM; Longhorn Vaccines and Diagnostics, LLC; Bethesda, Md.) has been shown to be compatible with commonly used extraction kits and to inactivate M. tuberculosis, while preserving the DNA for analysis. When transferred into PS-MTM, the cell wall of the bacilli is destroyed and inactivated, and the DNA is released into the solutions. In addition, the resulting mix provides for easy transportation of specimens at ambient temperature. PS-MTM samples spiked with M. tuberculosis in a dilution series was compared with a dilution series in PBS. PS-MTM increased the detection of M. tuberculosis by Xpert in dilutions with 100 or less cfu/ml over PBS samples, i.e. increasing Xpert efficiency.

Sixty-nine cough specimens were collected and submitted to the National Health Laboratory Services (NHLS) TB laboratory at Steve Biko Hospital in Pretoria, South Africa. As determined by visual inspection, 65 sample were judged to be of poor quality. As per NHLS definition of specimens and unfit for processing. Samples were selected that seemed to be salivary (including of less than 2 ml in volume (determined by measuring the height of liquid from the bottom of the sputum cup to the meniscus; one cm height represented approximately 1 ml of fluid in the screw-capped, wide-mouthed 50 ml cups for sputum collection used in local TB clinics), containing foreign particulate matter such as food particles, predominantly bloody, or.

Table 4 provides a break-down of the poor-quality categories, also showing that most of the specimens so classified were salivary in nature (50/65=76.9%).

TABLE 4
Specimen profile (n = 69) and definition of series
included in data analysis (n = 61)

| Specimens | Numbers | Percentages |
|---|---|---|
| Actually Collected | 69 | |
| Poor Quality | 65 | 94.2 of actual |
| Salivary | 50 | 76.9 of poor quality |
| Particulate Matter | 08 | 12.3 of poor quality |
| Bloody | 04 | 6.2 of poor quality |
| Nasal | 03 | 4.6 of poor quality |
| Acceptable | 04 | 6.2 of poor quality |

| Specimens Cultured | Number | Percentage |
|---|---|---|
| Actual (with MGIT and species ID) | 65 | |
| Positive for M. tuberculosis | 13 | 20 of actual |
| Positive for M. avium | 02 | |
| Positive for M. fortuitum | 01 | |
| No growth | 49 | |

| Process Step | Number |
|---|---|
| Submitted for Routine Xpert MTB/RIF Processing | 22 |
| Not Processed (quality criteria not met) | 43 |
| PS-MTM Processed by Xpert MTB/RIF | 65 |
| Positive | 50 |
| Negative | 15 |
| Error | 01 |

As part of routine NIILS laboratory processing, specimens were cultured for mycobacteria in MGIT and the species of isolates determined. M. tuberculosis organisms were grown from 13/65, M. avium from two, and M. fortuitum from one. Forty-nine specimens showed no growth. In parallel, all 65 specimens were also submitted for routine Xpert by the NHLS laboratory. As shown in Table 4, about one-third of specimens (22/65=33.8%) could not be assayed because of poor quality. Results from the remaining 43 specimens were recorded.

Xpert on samples collected in molecular transport medium was performed on all 65 specimens in the study, as follows: A flocked swab was swirled five times in each specimen cup (specimens not decontaminated) to collect a small quantity of sample (0.05 to 0.2 ml) for transfer into 1.5 ml PrimeStore-MTM (Longhorn Vaccines and Diagnostics, San Antonio, Tex.). The swab in PS-MTM was vortexed for 10 seconds to spin captured material into the solution, the swab was removed, and the solution left to stand at room temperature for 30 min to allow for lysing of cells and the release of DNA.5 Set-up and analysis of samples by Xpert MTB/RIF (Cepheid, USA) were performed as follows: From the PS-MTM tube, 0.7 ml specimen was transferred into 1.4 ml of Xpert MTB/RIF sample buffer (Cepheid, 2:1 ratio, v/v), vortexed 10-20 times and left to stand for 15 min at room temperature with intermittent shaking.6 From this mixture, 2 ml was added into a Xpert MTB/RIF Version 4 cartridge and analysed.

Of the 65 specimens processed, 64 returned a valid result and one returned an error message (Table 4).

RT-PCR amplification for M. tuberculosis detection was performed on all discrepant results between culture and Xpert (both PS-MTM Xpert and the NFILS routine Xpert results). PrimeMix MTB Complex (Longhorn Vaccines and Diagnostics, LLC; Bethesda, Md.), a mixture of primers, buffers, salts, and enzymes that targets a conserved region of the TB insertion sequence (IS) 611 0 region, was used according to methods described earlier. Real-time amplification was carried out in a final volume of 20 pL containing 15 μL PrimeMix and 5 extracted DNA using a LightCycler® 480 Instrument (Roche Life Science, Indiana, USA). The conditions for thermocycling were 95° C. for 5 min, followed by 40 cycles at 95° C. for 15 sec, and 60° C. for 32 sec. For instrument analysis, the 0.1 baseline threshold was used.

Samples were classified as positive if the cycle threshold (CT) value was below 38 indeterminate if CT was 38-40, and negative if no amplification signal was observed. For quantitative analysis, each sample was tested in duplicate to obtain an average CT. For extraction and real-time amplification positive and negative controls, M. tuberculosis reference strain H37Rv used water.

After excluding the results of specimens harboring non-tuberculous isolates (n=3) and of the specimen returning an error on Xpert processing of PS-MTM samples, the final data series included results for 61 of the 65 specimens.

Of the 61 cultured specimens included here, 13 (21.3%) were positive by culture, of which seven (53.8%) were also detected by routine Xpert, two were rated as negative, and four were not processed because of poor quality and/or insufficient specimen volume (Table 5). In addition, M. tuberculosis DNA was detected from three-of the entire negative specimens. PS-MTM Xpert identified 10/13 (76.9%) of the culture positives, including the same seven found positive by routine Xpert (Table 5).

TABLE 5
Detection of M. tuberculosis in low quality
cough specimens by PS-MTM samples by Xpert MTB/RIF
testing compared to liquid culture results

| | |
|---|---|
| PS-MTM Xpert | Culture |

| | Pos. | Neg. | Total |
|---|---|---|---|
| Pos. | 10 | 05 | 15 |
| Neg. | 03 | 43 | 46 |
| Total | 13 | 48 | 61 |

There were 48 culture negative poor-quality specimens included in the data set. PS-MTM Xpert was performed on all, including the 22 specimens for which routine Xpert were not done, and detected five positives. Routine Xpert detected three additional positives from the 26 specimens processed that were recorded as negative by PS-MTM Xpert assay and culture.

PS-MTM Xpert failed to detect 3/13 culture positive specimens. In order to elucidate the results further, we performed RT-PCR assays on all 11 specimens (PS-MTM or routine) discrepant to culture (positive or negative). RT-PCR confirmed all 11 as positive.

Overall, M. tuberculosis was grown in culture from 13/61 (21.3%) poor quality specimens and nucleic acid amplification tests (Xpert or RT-PCR) of PS-MTM samples detected the same 13 plus an additional 8 (i.e., 21/61 or 34.4%).

Previous studies on salivary or bloody specimens with a different transport medium/buffer presented to Xnert MTB/RIF also suggested enhanced detection of M. tuberculosis. Results with PS-MTM confirm the approach, but also indicate that RT-PCR detection was superior to Xpert and even culture. Sensitivity of detection is markedly increased.

RT-PCR positive to negative culture discrepancies might reflect non-viable bacilli being present in the specimen and therefore cannot be used for clinical decision-making. The proposal is for screening of poor-quality specimens sampled into PS-MTM and batch-assayed by RT-PCR, and positive results be followed-up with further clinical investigation of the patients involved. The alternative is for all poor-quality specimens to be rejected and follow-up sputum be collected for testing, adding unnecessary cost and time to making a diagnosis. First opportunities lost often translate into cases of tuberculosis being missed, with the consequence that transmission of tuberculosis continues.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, wherever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A rapid method for detecting genetic variation within a target sequence of a genome of an organism comprising
providing a pair of nucleic acid primers that span the target sequence, wherein the target sequence comprises a conserved region of the genome;
providing two nucleic acid probes, wherein each probe hybridizes to the target sequence, the sequence of a one probe differs from the sequence of an other probe by one nucleotide, and each probe is differentially labeled at each respective 5′-terminus and/or 3′-terminus;
combining the pair of nucleic acid primers and the two nucleic acid probes with the target sequence forming a mixture;
performing a polymerase chain reaction (PCR) of the mixture; detecting the labels; and
determining the presence of genetic variation in the target sequence by the differential quantity of each label detected.

2. The method of claim 1, wherein the organism comprises a bacterium, a virus, a fungus, or a mammal.

3. The method of claim 2, wherein the organism comprises a Mycobacteria.

4. The method of claim 3, wherein the target sequence comprises a segment of a gene or genome that confers drug resistance to the organism.

5. The method of claim 4, wherein the gene comprises a rpoB gene or a katG gene.

6. The method of claim 5, wherein the gene comprises the rpoB gene and the sequences of the probes differ at amino acid position 531 of the rpoB gene.

7. The method of claim 6, wherein the gene comprises the katG gene and the sequences of the probes differ at amino acid position 315 of the katG gene.

8. The method of claim 2, wherein the organism comprises an Influenza virus.

9. The method of claim 8, wherein the target sequence comprises a segment of a gene or genome that confers drug resistance to the organism.

10. The method of claim 9, wherein the gene is a protective antigen gene that confers drug resistance to baloxavir marboxil.

11. The method of claim 10, wherein the probes differ at amino acid position 38 of the protective antigen gene.

12. The method of claim 9, wherein the gene is a neuraminidase gene that confers drug resistance to oseltamivir.

13. The method of claim 12, wherein the neuraminidase gene is an N1 gene and the probes differ at amino acid position 275 of the N1 gene.

14. The method of claim 12, wherein the neuraminidase gene is an N2 gene and the probes differ at amino acid position 292 of the N2 gene.

15. The method of claim 1, wherein the conserved region is about 100 to about 300 nucleotides in length.

16. The method of claim 1, wherein the pair of nucleic acid primers each have a GC content of about 65%.

17. The method of claim 16, wherein the mixture contains a reducing agent.

18. The method of claim 17, wherein the reducing agent comprises DMSO or TCEP at a concentration of from about 0.01 mM to about 500 mM.

19. The method of claim 18, wherein the concentration is from about 1.0 mM to about 50 mM.

20. The method of claim 1, wherein the probes are differentially labeled with a fluorochrome.

21. The method of claim 20, wherein the fluorochrome comprises FAM, JOE, ROX, VIC, ABY, JUN, TAMRA, NED, TET, HEX, PET, or a combination thereof.

22. The method of claim 1, further comprising positive and/or negative controls of the PCR reaction.

23. The method of claim 1, wherein the PCR comprises qPCR.

24. The method of claim 23, wherein qPCR comprises a temperature cycling that provides for denaturation followed by annealing and extension.

25. The method of claim 24, wherein the temperature cycling comprises multiple cycles of from about 15° C. to about 25° C. followed by from about 50° C. to about 80° C.

26. The method of claim 1, wherein the one probe comprises a wild type target sequence.

27. The method of claim 26, wherein the label detected predominantly is the label associated with the 5-terminus of the one probe.

28. The method of claim 26, wherein the label detected predominantly is the label associated with the 5-terminus of the other probe.

29. The method of claim 26, wherein the labels associated with the 5-terminus of each probe are detected in substantially equal quantities.

30. The method of claim 1, wherein labels detected are predominantly the label associated with the 5′-terminus of each probe.

31. The method of claim 1, wherein labels detected are predominantly the label associated with the 3′-terminus of each probe.

32. The method of claim 1, which is performed in 8 hours or less.

33. The method of claim 32, which is performed in 4 hours or less.

34. The method of claim 33, which is performed in 2 hours or less.

35. The method of claim 1, wherein the organism is a mammal and the genetic variation comprises an allele associated with a genetic disease or disorder.

36. The method of claim 35, wherein the genetic disease or disorder comprises expression or absence of expression of an enzyme, immune system functioning, generation of a B cell or T cell response to an infection, or resistance or sensitivity to a drug.

37. The method of claim 1, wherein the method is performed simultaneously on multiple different mixtures.

38. The method of claim 37, wherein the organism is a Mycobacterium and the method detects multiple genetic variations.

39. The method of claim 38, wherein the multiple genetic variations detected indicate Mycobacterium with multiple drug resistance.

40. The method of claim 38, wherein the multiple genetic variations detected indicate multiple different Mycobacterium each with a different drug resistance profile.

41. The method of claim 1, wherein the diseased cells are indicative of the presence of a pathogen or the presence of cancerous tissue.

42. The method of claim 2, wherein the pathogen comprises one or more of a virus, a bacterium, a fungus or a parasite.

43. The method of claim 3, wherein the virus is one or more of a DNA virus, an RNA virus, a positive or negative single-strand virus, a double strand virus, an orthomyxovirus, a paramyxovirus, a retrovirus, an Arbovirus, a Zika virus, a flavivirus, a filovirus, a lentivirus, an influenza virus, a human immunodeficiency virus, a hepatitis virus, or an ebola virus.

44. The method of claim 3, wherein the bacterium is Mycobacterium tuberculosis, Plasmodium falciparum, Francisella tularensis, Yersinia pestis, or Vibrio cholera.

45. The method of claim 1, wherein the genome is obtained from bodily fluid and/or tissue of the patient.

46. The method of claim 1, where the biological sample is provided in a molecular transport medium and the molecular transport medium contains a chaotrope, a detergent, a reducing agent, a chelator, a buffer, and an alcohol, together present in an amount sufficient to lyse cells, denature proteins, inactivate nucleases, kill pathogens, and not degrade nucleic acid.

47. The method of claim 1, wherein the mutations conferring drug resistance are mutations conferring resistance to an antibiotic or a chemotherapy.

48. The method of claim 1, wherein the quantitative polymerase chain reaction is carried out in an aqueous mix comprising: a polymerase and optionally a reverse transcriptase; a mix of deoxynucleotide tri phosphates comprising about equivalent amounts of dATP, dCTP, dGTP and dTTP, a chelating agent, an osmolarity agent, an albumin, a magnesium salt; and a buffer.
